(19) United States
(12) Patent Application Publication
(10) Pub. No.: US 2005/0112063 A1
(43) Pub. Date: May 26, 2005

(54) Methods for inhibiting vascular permeability

(76) Inventors: Shay Soker, Greensboro, NC (US); Mark W. Kieran, Newton, MA (US)

Correspondence Address: David S. Resnick;NIXON PEABODY LLP, 100 Summer Street, Boston, MA, 02110-2131, US

(21) Appl. No.: 10/962,901

(22) Filed: Oct. 12, 2004

Related U.S. Application Data
(63) Continuation of application No. PCT/US03/11265, filed on Apr. 11, 2003.
(60) Provisional application No. 60/371,841, filed on Apr. 11, 2002.

(51) Int. Cl. A61K 49/00

(52) U.S. Cl. 424/9.2

(57) ABSTRACT

The present invention relates to methods for decreasing or inhibiting disorders associated with vascular hyperpermeability and to methods of screening for compounds that affect permeability, angiogenesis and stabilize tight junctions. In one aspect of the present invention there is provided a method of decreasing or inhibiting vascular hyperpermeability in an individual in need of such treatment. The method includes administering to the individual an effective amount of an antiangiogenic compound selected from the group consisting of endostatin, thrombospondin, angiostatin, tumstatin, arrestin, recombinant EPO and polymer conjugated TNP-470. Other antiangiogenic compounds are disclosed herein.

### GOVERNMENT FUNDING

This invention was made with government support under CA45548 awarded by the National Institutes of Health. The government has certain rights in the invention.

### FIELD OF THE INVENTION

The present invention relates to methods for decreasing or inhibiting disorders associated with vascular hyperpermeability and to methods of screening for compounds that affect permeability, angiogenesis and stabilize tight junctions.

### BACKGROUND OF THE INVENTION

Vascular hyperpermeability has been implicated in numerous pathologies including vascular complications of diabetes, pulmonary hypertension and various edemas, and has been rendered responsible for decreasing efficacy of anti-cancer therapies due to loss of endogenous angiogenesis inhibitors into the urine. For instance, a complication of diabetes, diabetic retinopathy is a leading cause of blindness that affects approximately 25% of the estimated 16 million Americans with diabetes. It is believed that diabetic retinopathy is induced by hypoxia in the retina as a result of hyperglycemia.

The degree of diabetic retinopathy is highly correlated with the duration of diabetes. There are two kinds of diabetic retinopathy. The first, non-proliferative retinopathy, is the earlier stage of the disease characterized by increased capillary permeability, microaneurysms, hemorrhages, exudates, and edema. Most visual loss during this stage is due to the fluid accumulating in the macula, the central area of the retina. This accumulation of fluid is called macular edema, and can cause temporary or permanent decreased vision. The second category of diabetic retinopathy is called proliferative retinopathy and is characterized by abnormal new vessel formation, which grows on the vitreous surface or extends into the vitreous cavity. Neovascularization can be very damaging because it can cause bleeding in the eye, retinal scar tissue, diabetic retinal detachments, or glaucoma, any of which can cause decreased vision or blindness.

Current treatment of non-proliferative retinopathy includes intensive insulin therapy to achieve normal glycemic levels in order to delay further progression of the disease, whereas the current treatment of proliferative retinopathy involves panretinal photocoagulation and vitrectomy. The treatment of non-proliferative retinopathy, while valid in theory, is mostly ineffective in practice because it usually requires considerable modification in the lifestyle of the patients, and many patients find it very difficult to maintain the near-normal glycemic levels for a time sufficient to slow and reverse the progression of the disease. Thus, the current treatment of non-proliferative retinopathy only delays the progression of the disease and cannot be applied effectively to all patients who require it.

Another complication of diabetes, diabetic nephropathy is the dysfunction of the kidneys and the most common cause of end-stage renal disease in the USA. It is a vascular complication that affects the glomerular capillaries of the kidney and reduces the kidney's filtration ability. Nephropathy is first indicated by the appearance of hyperfiltration and then microalbuminuria. Heavy proteinuria and a progressive decline in renal function precede end-stage renal disease. It is believed that hyperglycemia causes glycosylation of glomerular proteins, which may be responsible for mesangial cell proliferation and matrix expansion and vascular endothelial damage. Typically before any signs of nephropathy appear, retinopathy has usually been diagnosed.

Early treatment of nephropathy can attenuate disease progression. Currently, aggressive treatment is indicated including protein, sodium and phosphorus restriction diet, intensive glycemic control, ACE inhibitors (e.g., captopril) and/or nondihydropyridine calcium channel blockers (diltiazem and verapamil), C-peptide and somatostatin are also used. The treatment regimen for early-stage nephropathy comprising dietary and glycemic restrictions is less effective in practice than in theory due to difficulties associated with patient compliance. Renal transplant is usually recommended to patients with end-stage renal disease due to diabetes. Survival rate at 5 years for patients receiving a transplant is about 60% compared with only 2% for those on dialysis. Renal allograft survival rate is greater than 85% at 2 years.

Vascular hyperpermeability plays an important role in complications of nephrotic syndrome. Nephrotic syndrome is a condition characterized by massive edema (fluid accumulation), heavy proteinuria (protein in the urine), hypoalbuminemia (low levels of protein in the blood), and susceptibility to infections. Nephrotic syndrome results from damage to the kidney's glomeruli. Glomeruli are tiny blood vessels that filter waste and excess water from the blood. The damaged glomeruli are characterized by hyperpermeability. Nephrotic syndrome can be caused by glomerulonephritis, diabetes mellitus, or amyloidosis. Presently, prevention of nephrotic syndrome relies on controlling these diseases.

One serious complication of nephrotic syndrome is thrombosis (blood clotting), especially in the brain. The loss of plasma proteins due to hyperpermeability of the glomeruli in patients with nephrotic syndrome leads to a reduced concentration of Antithrombin III (ATIII). ATIII is one of the most important regulators of the coagulation system. Low levels of ATIII in the blood means a great and well established risk for thrombotic complications, especially blood clots in the brain. Decreasing permeability of glomeruli would prevent thrombosis.

Vascular hyperpermeability has also been found to play a role in pathophysiology of nephrotic edema in human primary glomerulonephritis, such as idiopathic nephrotic syndrome (INS). It is believed that vascular hyperpermeability in nephrotic edema is related to the release of vascular permeability factor and other cytokines by immune cells. See Rostoker et al., Nephron 85:194-200 (2000).

Pulmonary hypertension is a rare blood vessel disorder of the lung in which the pressure in the pulmonary artery (the blood vessel that leads from the heart to the lungs) rises above normal levels and may become life threatening. Pulmonary hypertension has been historically chronic and incurable with a poor survival rate. Recent data indicate that the length of survival is continuing to improve, with some patients able to manage the disorder for 15 to 20 years or longer.

Pulmonary hypertension is caused by alveolar hypoxia, which results from localized inadequate ventilation of well-perfused alveoli or from a generalized decrease in alveolar ventilation. Treatment of pulmonary hypertension usually involves continuous use of oxygen. Pulmonary vasodilators (e.g., hydralazine, calcium blockers, nitrous oxide, prostacyclin) have not proven effective. Lung transplant is typically recommended to patients who do not respond to therapy.

It is well known that the members of the vascular endothelial growth factor (VEGF) family induce vascular permeability. Compounds designed to inhibit the activity of VEGF, including anti-VEGF antibodies, anti-VEGF receptor antagonists and small molecules that inhibit receptor tyrosin kinase, activity should also inhibit VEGF induced vascular permeability. However, these compounds would have no effect on vascular permeability that is VEGF-independent. It would be desirable to have a method to inhibit both VEGF-independent and dependent vascular permeability and thus provide alternatives to treating disorders whose pathology is associated with vascular hyperpermeability, such as non-proliferative diabetic retinopathy, diabetic nephropathy, nephrotic syndrome, pulmonary hypertension and various edemas.

### SUMMARY OF THE INVENTION

In one aspect of the present invention there is provided a method of decreasing or inhibiting vascular hyperpermeability in an individual in need of such treatment. The method includes administering to the individual an effective amount of an antiangiogenic compound selected from the group consisting of endostatin, thrombospondin, angiostatin, tumstatin, arrestin, recombinant EPO and polymer conjugated TNP-470. Other antiangiogenic compounds are disclosed herein.

An “antiangiogenic compound”, as used herein, is a compound capable of inhibiting the formation of blood vessels. The disease associated with vascular permeability for treatment with the present invention includes vascular complications of diabetes such as non-proliferative diabetic retinopathy and diabetic nephropathy, nephrotic syndrome, pulmonary hypertension, burn edema, tumor edema, brain tumor edema, IL-2 therapy-associated edema, and other edema-associated diseases. The method of the invention can be used to prevent the leakage from blood vessels of natural angiogenesis inhibitors.

In yet another aspect of the present invention there is provided a method of treating and/or preventing a disease associated with vascular hyperpermeability in an individual in need of such treatment. The method involves administering to the individual an effective amount of a compound capable of increasing cell-cell contacts by stabilizing tight junction complexes and increasing contact with the basement membrane. Effective compounds are, for example, endostatin, thrombospondin, angiostatin, tumstatin, arrestin, recombinant EPO and polymer conjugated TNP-470. In certain embodiments, it may be desirable to conjugate the antiangiogenic agent with a polymer. An HPMA copolymer is preferred.

In a further aspect of the invention there is provided a method of screening for compounds that stabilize tight junction complexes. The method involves culturing endothelial cells in the presence of a test compound, incubating with the cultured endothelial cells expressing junction proteins, and assessing whether the test compound stabilized the tight junction complexes. The assessment of stabilization of a tight junction protein can be readily performed by immunostaining for that protein and visualized under fluorescent microscopy. Intense cell-boundary staining is indicative of a compound that stabilizes the tight junction protein, and, therefore, is indicative of an anti-permeability and/or an anti-angiogenic activity which can be further tested for such activity. The tight junction proteins contemplated by the present invention include integral membrane proteins, cytoplasmic proteins, and proteins associated with tight junctions. More particularly, the tight junction proteins include occludin, claudin, zonula occludens (ZO)-1, -2, -3, catenins, VE cadherin, cingulin and p130.

In a further aspect of the invention there is provided a method of screening for compounds that affect vascular permeability. The method involves assaying endothelial cells on a permeable substrate (e.g., a collagen coated inserts of “Transwells”), contacting the assay with a test compound, treating the assay with a mixture of markers (e.g., FITC label) and permeability-inducing agents (e.g., vascular endothelial growth factor (VEGF) and platelet-activating factor (PAF) among others), and measuring the amount of marker to travel through the substrate. The test compound with antipermeability properties would cause the marker to diffuse slower compare to the control and to permeability-inducing agents.

In another aspect of the present invention there is provided a method for assessing bioeffectiveness of an antiangiogenic compound in a patient being treated with such compound. The method involves administering to the patient an intradermal/subcutaneous injection of histamine before treating the patient with the antiangiogenic compound and measuring a histamine-induced local edema. Thereafter, treating the patient with the antiangiogenic compound, and again administering to said patient an intradermal/subcutaneous injection of histamine subsequent to treating the patient with the antiangiogenic compound and measuring the histamine-induced local edema. A decrease in the measurement of the histamine-induced local edema compared to that seen before the treatment with the antiangiogenic compound indicates that the compound is bioeffective.

The present invention also provides an alternative method for assessing bioeffectiveness of an antiangiogenic compound in a patient being treated with such compound. The method involves measuring a level of a protein in a bodily fluid of the patient (e.g., blood or urine) before treating the patient with the antiangiogenic compound, then, treating the patient with the antiangiogenic compound and measuring the level of the protein in the bodily fluid of the patient. A decrease in the level of the protein in the bodily fluid compare to the pre-treatment level indicates that the compound inhibits vascular permeability and is bioeffective.

Finally, the present invention provides an article of manufacture which includes packaging material and a pharmaceutical agent contained within the packaging material. The packaging material includes a label which indicates said pharmaceutical may be administered, for a sufficient term at an effective dose, for treating and/or preventing a disease associated with vascular permeability. The pharmaceutical agent is selected from the group consisting of endostatin, thrombospondin, angiostatin, tumstatin, arrestin, recombinant EPO and polymer conjugated TNP-470. The disease associated with vascular permeability includes, but not limited to, vascular complications of diabetes such as non-proliferative diabetic retinopathy and diabetic nephropathy, nephrotic syndrome, pulmonary hypertension, burn edema, tumor edema, brain tumor edema, IL-2 therapy-associated edema, and other edema-associated diseases.

Unless otherwise defined, all technical and scientific terms used herein have the same meaning as commonly understood by one of ordinary skill in the art. Although methods and materials similar or equivalent to those described herein can be used in the practice or testing of the invention, the preferred methods and materials are described below. All publications, patent applications, patents and other references mentioned herein are incorporated by reference. In addition, the materials, methods and examples are illustrative only and not intended to be limiting. In case of conflict, the present specification, including definitions, controls.

### BRIEF DESCRIPTION OF THE DRAWINGS

The accompanying drawings, which are incorporated in and constitute a part of this specification, illustrate embodiments of the invention and, together with the description, serve to explain the objects, advantages, and principles of the invention.

FIG. 1 is a quantitative analysis of Evans Blue dye extravasation showing lower skin capillary permeability of the antiangiogenic factor-treated mice and indicated the weak permeability-inducing effect of VEGF in these mice.

FIG. 2 is a quantitative analysis of Evans Blue dye extravasation showing lower skin capillary permeability of the endostatin-treated mice compared with control and the lack of PAF-induced hyperpermeability in these mice.

FIG. 3 is a quantitative analysis of skin vessel permeability of saline and endostatin-treated mice, during a contiguous period of time, and skin vessel permeability in response to PAF injection.

FIG. 4 illustrates that endostatin treatment significantly reduces the diffusion of large molecules through the endothelial cell monolayer.

FIGS. 5 and 6 show kinetics of the diffusion process using 10 kDa dextran (FIG. 5) and 70 kDa dextran (FIG. 6).

FIGS. 7A-7C show the effects of conjugated and free TNP-470 on liver regeneration after hepatectomy compare to control.

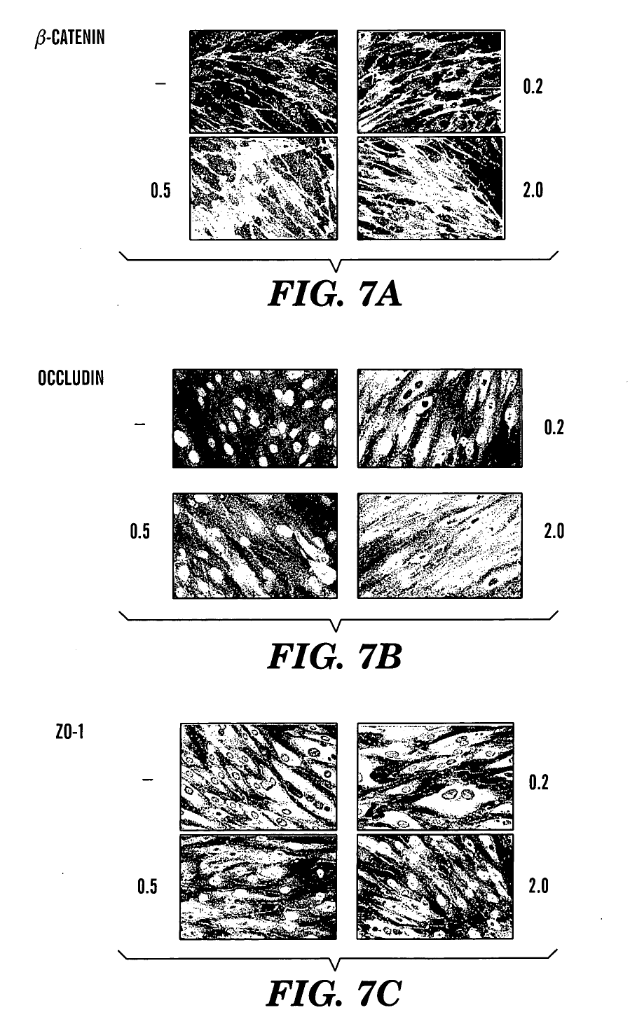

FIGS. 8A-8E show that free and polymer conjugated TNP-470 prevents VEGF, PAF and histamine-induced vascular leakage compare to control in the miles assay.

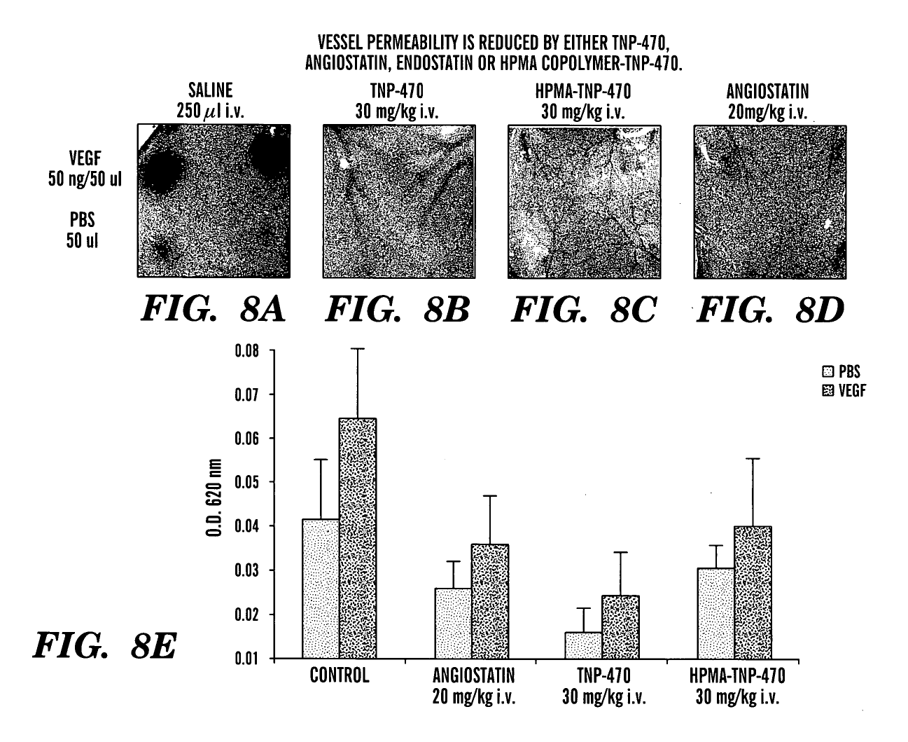

FIGS. 9A-9D show that the “indirect” angiogenesis inhibitors, Thalidomide and Herceptin, have no effect on vessel permeability.

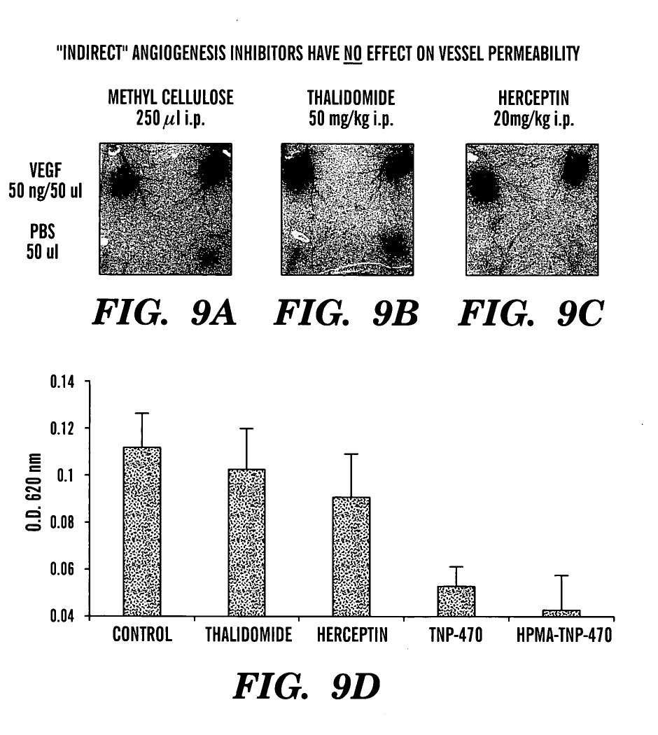

FIG. 10 shows the permeability effects in SCID mice bearing A2058 human melanoma treated for 3-5 days with angiostatin, TNP-470 and polymer conjugated TNP-470 prior to the Miles assay.

FIG. 11 shows bovine capillary endothelial (BCE) cells treated with TNP-470 for 3 days and stained with antibody to the tight junction protein ZO-1.

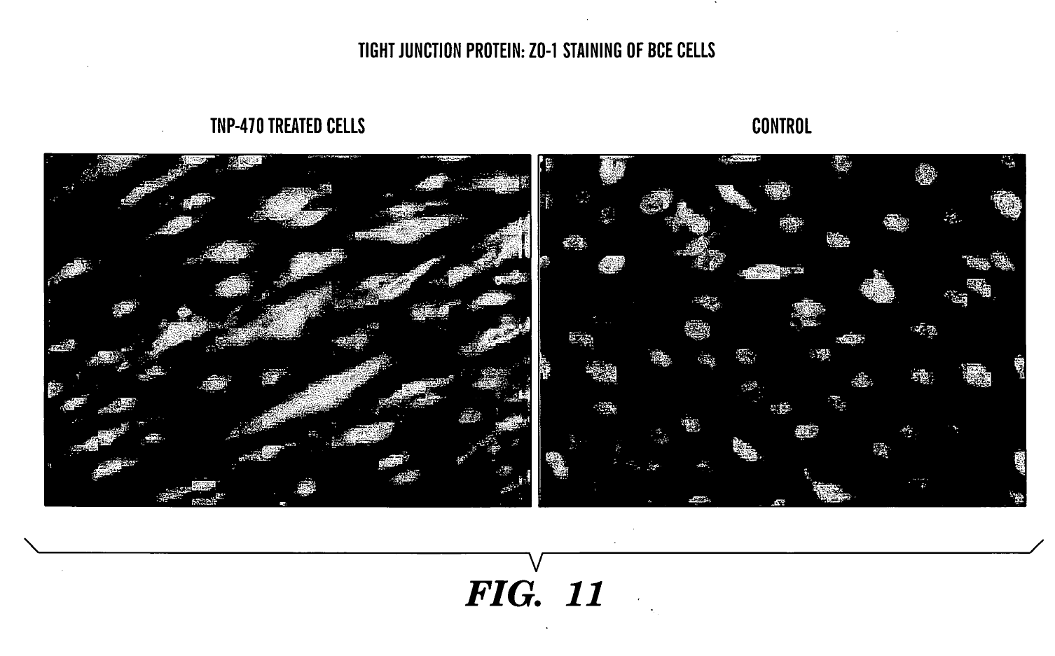

FIG. 12 shows the relative weight of the lungs following treatment with TNP-470 for 3 days compared to control lungs after induction of edema with IL-2 i.m. administration and control normal lungs. As shown in the graph, TNP-470 reduces pulmonary edema.

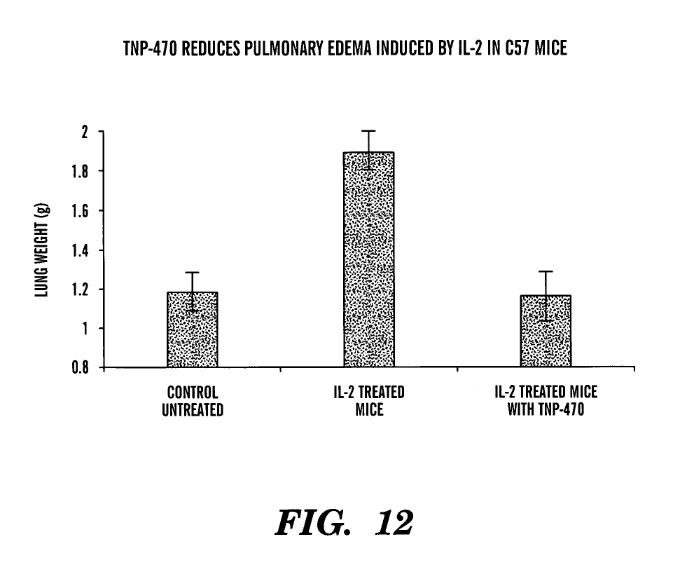

FIG. 13 shows the results in the Miles assay in SCID mice bearing A 2058 human melanoma treated for 5 days with endostatin.

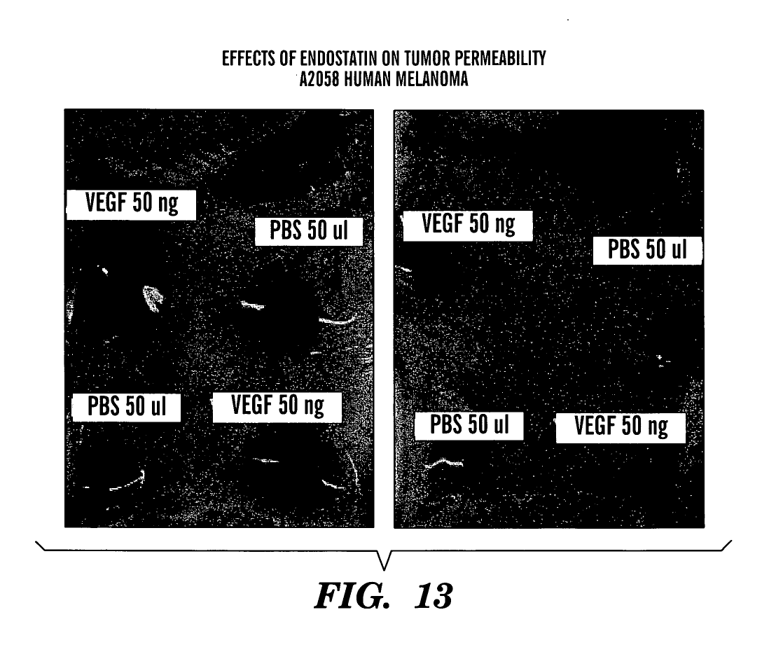

### DETAILED DESCRIPTION

We demonstrated in a mouse model that treatment with endostatin resulted in a significantly lower capillary leakage following intradermal injection of permeability-inducing agents (e.g., VEGF and platelet-activating factor (PAF)) compared with saline treated mice. These results suggest that the anti-tumor activity of endostatin might be explained in part by its anti-blood vessel permeability activity. Blood vessel permeability is associated with other diseases besides cancer such as vascular complications of diabetes such as diabetic retinopathy and nephropathy, nephrotic syndrome, vascular hypertension, burn edema, tumor edema, brain tumor edema, IL-2 therapy-associated edema, and other edema-associated diseases. Thus, molecules that display anti-angiogenic activity, such as endostatin, can be used to prevent and treat pathologic blood vessel hyperpermeability in addition to their use in anti-cancer therapy. Such molecules may also be used to prevent the loss of endogenous angiogenic inhibitors or chemotherapeutic agents into the urine and thus are useful in the treatment of diseases or disorders involving abnormal angiogenesis including cancer.

In one aspect of the present invention there is provided a method of decreasing or inhibiting vascular hyperpermeability in an individual in need of such treatment. The method involves administering to the individual an effective amount of an antiangiogenic compound selected from the group consisting of endostatin, thrombospondin, angiostatin, tumstatin, arrestin, recombinant EPO, and polymer conjugated TNP-470. Preferably, the polymer is a HPMA copolymer.

Other angiogenesis inhibitors useful in the present invention include Taxane and derivatives thereof; interferon alpha, beta and gamma; IL-12; matrix metalloproteinases (MMP) inhibitors (e.g.: COL3, Marimastat, Batimastat); EMD121974 (Cilengitide); Vitaxin; Squalamin; Cox2 inhibitors; PDGFR inhibitors (e.g., Gleevec); EGFR1 inhibitors (e.g., ZD1839 (Iressa), DSI774, SI1033, PKI166, IMC225 and the like); NM3; 2-ME2; Bisphosphonate (e.g., Zoledronate).

Taxane (paclitaxel) derivatives are disclosed in WO01017508, the disclosure of which is incorporated herein by reference.

Examples of inhibitors of matrix metalloproteinases include, but are not limited to, tetracycline derivatives and other non-peptidic inhibitors such as AG3340 (from Agouron), BAY 12-9566 (from Bayer), BMS-275291 (from Bristol-Myers Squibb) and CGS 27023 Å (from Novartis) or the peptidomimetics marimastat and Batimastat (from British Biotech), and the MMP-3 (stromelysin-1) inhibitor, Ac-RCGVPD-NH2 available from Calbiochem (San Diego, Calif.). See Hidalgo et al. 2001. J. Natl. Can. Inst. 93: 178-93 for a review of MMP inhibitors in cancer therapy.

As used herein the term “COX-2 inhibitor” refers to a non-steroidal drug that relatively inhibits the enzyme COX-2 in preference to COX-1. Preferred examples of COX-2 inhibitors include, but are no limited to, celecoxib, parecoxib, rofecoxib, valdecoxib, meloxicam, and etoricoxib.

In accordance with the present invention, fumagilin analogs other than TNP-470 may also be used. Such analogs include those disclosed in U.S. Pat. Nos. 5,180,738 and 4,954,496.

The antiangiogenic agent may be linked to a water soluble polymer having a molecular weight in the range of 100 Da to 800 kD. The components of the polymeric backbone may comprise acrylic polymers, alkene polymers, urethanepolymers, amide polymers, polyimines, polysaccharides and ester polymers. Preferably the polymer is synthetic rather than being a natural polymer or derivative thereof. Preferably the backbone components comprise derivatised polyethyleneglycol and poly(hydroxyalkyl(alk)acrylamide), most preferably amine derivatised polyethyleneglycol or hydroxypropyl(meth)acrylamide-methacrylic acid copolymer or derivative thereof. A preferred molecular weight range is 15 to 40 kD.

The antiangiogenic agent and the polymer are conjugated by use of a linker, preferably a cleavable peptide linkage. Most preferably, the peptide linkage is capable of being cleaved by preselected cellular enzymes. Alternatively, an acid hydrolysable linker could comprise an ester or amide linkage and be for instance, a cis-aconityl linkage. A pH sensitive linker may also be used.

Cleavage of the linker of the conjugate results in release of an active antiangiogenic agent. Thus the antiangiogenic agent must be conjugated with the polymer in a way that does not alter the activity of the agent. The linker preferably comprises at least one cleavable peptide bond. Preferably the linker is an enzyme cleavable oligopeptide group preferably comprising sufficient amino acid units to allow specific binding and cleavage by a selected cellular enzyme. Preferably the linker is at least two amino acids long, more preferably at least three amino acids long.

Preferred polymers for use with the present invention are HPMA copolymers with methacrylic acid with pendent oligopepticle groups joined via peptide bonds to the methacrylic acid with activated carboxylic terminal groups such as paranitrophenyl derivatives.

In a preferred embodiment the polymeric backbone comprises a hydroxyalkyl(alk)acrylamide methacrylamide copolymer, most preferably a copolymer of N-(2-hydroxypropyl)methacrylamide (HPMA) copolymer. Such polymers and methods of conjugation are disclosed in WO 01/36002.

A disease associated with vascular permeability for treatment with the present invention includes vascular complications of diabetes such as non-proliferative diabetic retinopathy and nephropathy, nephrotic syndrome, pulmonary hypertension, burn edema, tumor edema, brain tumor edema, IL-2 therapy-associated edema, and other edema-associated diseases.

Tight junctions regulate endothelial cell permeability and create an intramembrane diffusion fence. Tight junctions form discrete sites of fusion between the outer plasma membrane of adjacent cells. The tight junctions are complexes of molecules that build, associated with, or regulate the tight junction function. The junctions are composed of three regions: the integral membrane proteins, including, but not limited to, occludin and claudin; the cytoplasmic proteins, including, but not limited to, zonula occludens (ZO)-1, -2, -3; and proteins associated with tight junctions, including, but not limited to, catenins, cingulin and p130. Recent studies have shown that VEGF interferes with tight junction assembly via induction of rapid phosphorylation of tight junction proteins occludin and ZO-1, resulting in dislocation of these proteins from the cell membrane. VEGF was also shown to decrease the expression of occludin. We show in the examples below that interference with or destabilization of tight junction proteins increases vascular permeability and ultimately causes hyperpermeability. Therefore, stabilization of the tight junction proteins using compounds which inhibit endothelial cell proliferation and migration in vitro or otherwise repress tumor growth would be useful in the treatment or prevention of diseases associated with vascular hyperpermeability.

Compounds such as endostatin, thrombospondin, angiostatin, tumstatin, arrestin, recombinant EPO, and TNP-470 are widely available commercially. Those compounds that are not commercially available can be readily prepared using organic synthesis methods known in the art.

Whether or not a particular compound, in accordance with the present invention, can treat or prevent diseases associated with hyperpermeability can be determined by its effect in the mouse model as shown in the Examples below. Compounds capable of preventing or treating non-proliferative diabetic retinopathy can be tested by in vitro studies of endothelial cell proliferation and in other models of diabetic retinopathy, such as Streptozotocin. In addition, color Doppler imaging can be used to evaluate the action of a drug in ocular pathology (Valli et al., Ophthalmologica 209(13): 115-121 (1995)). Color Doppler imaging is a recent advance in ultrasonography, allowing simultaneous two-dimension imaging of structures and the evaluation of blood flow. Accordingly, retinopathy can be analyzed using such technology.

The compounds useful in the prevention and treatment methods of the present invention can be administered in accordance with the present inventive method by any suitable route. Suitable routes of administration include systemic, such as orally or by injection or topical. The manner in which the therapeutic compound is administered is dependent, in part, upon whether the treatment of a disease associated with vascular hyperpermeability, including non-proliferative retinopathy is prophylactic or therapeutic. For example, the manner in which the therapeutic compound is administered for treatment of retinopathy is dependent, in part, upon the cause of the retinopathy. Specifically, given that diabetes is the leading cause of retinopathy, the effective compound can be administered preventatively as soon as the pre-diabetic retinopathy state is detected.

Thus, to prevent non-proliferative retinopathy that can result from diabetes, the effective compound is preferably administered systemically, e.g., orally or by injection. To treat non-proliferative diabetic retinopathy, the effective compound can be administered systemically, e.g., orally or by injection, or intraocularly. Other routes such as periocular (e.g., subTenon's), subconjunctival, subretinal, suprachoroidal and retrobulbar can also be used in the methods of the present invention. The effective compound is preferably administered as soon as possible after it has been determined that an individual is at risk for retinopathy (preventative treatment) or has begun to develop retinopathy (therapeutic treatment). Treatment will depend, in part, upon the particular effective compound used, the amount of the effective compound administered, the route of administration, and the cause and extent, if any, of retinopathy realized.

One skilled in the art will appreciate that suitable methods of administering an effective compound, which is useful in the present inventive method, are available. Although more than one route can be used to administer the effective compound, a particular route can provide a more immediate and more effective reaction than another route. Accordingly, the described routes of administration are merely exemplary and are in no way limiting.

The dose of the effective compound administered to an individual, particularly a human, in accordance with the present invention should be sufficient to effect the desired response in the animal over a reasonable time frame. One skilled in the art will recognize that dosage will depend upon a variety of factors, including the strength of the particular compound employed, the age, condition or disease state (e.g., the amount of the retina about to be affected or actually affected by retinopathy), and body weight of the individual. The size of the dose also will be determined by the route, timing and frequency of administration as well as the existence, nature, and extent of any adverse side effects that might accompany the administration of a particular compound and the desired physiological effect. It will be appreciated by one of ordinary skill in the art that various conditions or disease states, in particular, chronic conditions or disease states, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. The present inventive method will typically involve the administration of from about 1 mg/kg/day to about 500 mg/kg/day, preferably from about 10 mg/kg/day to about 200 mg/kg/day, if administered systemically. Intraocular administration typically will involve the administration of from about 0.1 mg total to about 5 mg total, preferably from about 0.5 mg total to about 1 mg total.

Compositions for use in the present inventive method preferably comprise a pharmaceutically acceptable carrier and an amount of a compound sufficient to treat or prevent diseases associated with vascular hyperpermeability and non-proliferative retinopathy. The carrier can be any of those conventionally used and is limited only by chemico-physical considerations, such as solubility and lack of reactivity with the compound, and by the route of administration. It will be appreciated by one of ordinary skill in the art that, in addition to the following described pharmaceutical compositions, the compound used in the methods of the present invention can be formulated as polymeric compositions, inclusion complexes, such as cyclodextrin inclusion complexes, liposomes, microspheres, microcapsules and the like (see, e.g., U.S. Pat. Nos. 4,997,652, 5,185,152 and 5,718,922).

The effective compound used in the present invention can be formulated as a pharmaceutically acceptable acid addition salt. Examples of pharmaceutically acceptable acid addition salts for use in the pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, and arylsulphonic, for example p-toluenesulphonic, acids.

The pharmaceutically acceptable excipients described herein, for example, vehicles, adjuvants, carriers or diluents, are well-known to those who are skilled in the art and are readily available to the public. It is preferred that the pharmaceutically acceptable carrier be one which is chemically inert to the compound used and one which has no detrimental side effects or toxicity under the conditions of use.

The choice of excipient will be determined in part by the particular compound, as well as by the particular method used to administer the composition. Accordingly, there is a wide variety of suitable formulations of the pharmaceutical composition of the present invention. The following formulations are merely exemplary and are in no way limiting.

Injectable formulations are among those that are preferred in accordance with the present inventive method. The requirements for pharmaceutically effective carriers for injectable compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986)). It is preferred that such injectable compositions be administered intramuscularly, intravenously, or intraperitoneally.

Topical formulations are well-known to those of skill in the art. Such formulations are suitable in the context of the present invention for application to the skin. The use of patches, corneal shields (see, e.g., U.S. Pat. No. 5,185,152), and ophthalmic solutions (see, e.g., U.S. Pat. No. 5,710,182) and ointments, e.g., eye drops, is also within the skill in the art.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The effective compound for use in the methods of the present invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride, with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral.

Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metals, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-p-aminopropionates, and 2-alkyl-imidazoline quaternary ammonium salts, and (e) mixtures thereof.

The parenteral formulations will typically contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17.

The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Such compositions can be formulated as intraocular formulations, sustained-release formulations or devices (see, e.g., U.S. Pat. No. 5,378,475). For example, gelantin, chondroitin sulfate, a polyphosphoester, such as bis-2-hydroxyethyl-terephthalate (BHET), or a polylactic-glycolic acid (in various proportions) can be used to formulate sustained-release formulations. Implants (see, e.g., U.S. Pat. Nos. 5,443,505, 4,853,224 and 4,997,652), devices (see, e.g., U.S. Pat. Nos. 5,554,187, 4,863,457, 5,098,443 and 5,725,493), such as an implantable device, e.g., a mechanical reservoir, an intraocular device or an extraocular device with an intraocular conduit (e.g., 100 mu-1 mm in diameter), or an implant or a device comprised of a polymeric composition as described above, can be used.

The present inventive method also can involve the co-administration of other pharmaceutically active compounds. By “co-administration” is meant administration before, concurrently with, e.g., in combination with the effective compound in the same formulation or in separate formulations, or after administration of the effective compound as described above. For example, corticosteroids, e.g., prednisone, methylprednisolone, dexamethasone, or triamcinalone acetinide, or noncorticosteroid anti-inflammatory compounds, such as ibuprofen or flubiproben, can be co-administered. Similarly, vitamins and minerals, e.g., zinc, anti-oxidants, e.g., carotenoids (such as a xanthophyll carotenoid like zeaxanthin or lutein), and micronutrients can be co-administered. Other various compounds that can be co-administered include sulphonylurea oral hypoglycemic agent, e.g., gliclazide (non-insulin-dependent diabetes), halomethyl ketones, anti-lipidemic agents, e.g., etofibrate, chlorpromazine and spinghosines, aldose reductase inhibitors, such as tolrestat, sorbinil or oxygen, and retinoic acid and analogues thereof (Burke et al., Drugs of the Future 17(2): 119-131 (1992); and Tomlinson et al., Pharmac. Ther. 54: 151-194 (1992)). Those patients that exhibit systemic fluid retention, such as that due to cardiovascular or renal disease and severe systemic hypertension, can be additionally treated with diuresis, dialysis, cardiac drugs and antihypertensive agents.

In yet another aspect of the invention there is provided a method of screening for compounds that stabilize tight junction proteins. The method involves culturing endothelial cells in the presence of a test compound, contacting the cultured endothelial cells with a tight junction protein, and assessing whether the test compound stabilized the tight junction protein. The compound that stabilizes the tight junction protein is indicative of an anti-permeability and/or an anti-angiogenic compound. The tight junction protein contemplated by the present invention includes integral membrane proteins, cytoplasmic proteins, and proteins associated with tight junctions. More particularly, the tight junction proteins include occludin, claudin, zonula occludens (ZO)-1, -2, -3, catenins, cingulin and p130. One embodiment of the method of screening for compounds that stabilize tight junction proteins is described in the Examples section below.

In a further aspect of the invention there is provided a method of screening for compounds that affect vascular permeability. The method, one embodiment of which is described below in the Examples section of the application, involves assaying endothelial cells on a permeable substrate (e.g., a collagen coated inserts of “Transwells”), contacting the assay with a test compound (e.g., an antiangiogenic compound such as endostatin), treating the assay with a marker (e.g., FITC label) and a permeability-inducing agent (e.g., vascular endothelial growth factor (VEGF) and platelet-activating factor (PAF) among others), and measuring the rate of diffusion of the marker compare to control. Compounds that are found to affect vascular permeability can be further tested for anti-tumor activity using existing methods.

In another aspect of the present invention there is provided a method for assessing bioeffectiveness of an antiangiogenic compound in a patient being treated with such compound. The method involves administering to the patient an intradermal injection of histamine before treating the patient with the antiangiogenic compound and measuring a histamine-induced local edema. Then, treating the patient with the antiangiogenic compound, and again administering to said patient an intradermal injection of histamine subsequent to treating the patient with the antiangiogenic compound and measuring the histamine-induced local edema. A decrease in the measurement of the histamine-induced local edema compared to that seen before the treatment with the antiangiogenic compound indicates that the compound is bioeffective.

The present invention also provides an alternative method for assessing a bioeffectiveness of an antiangiogenic compound in a patient being treated with such compound. It has been observed that patients suffering from diseases associated with vascular hyperpermeability have higher protein levels in the urine compare to a control group. The method involves measuring a level of a protein in a bodily fluid of the patient (e.g., blood or urine) before treating the patient with the antiangiogenic compound, then, treating the patient with the antiangiogenic compound and measuring the level of the protein in the bodily fluid of the patient. A decrease in the level of the protein in the bodily fluid compare to the pre-treatment level indicates that the compound inhibits vascular permeability and is bioeffective.

Finally, the present invention provides an article of manufacture which includes packaging material and a pharmaceutical agent contained within the packaging material. The packaging material includes a label which indicates said pharmaceutical may be administered, for a sufficient term at an effective dose, for treating and/or preventing a disease associated with vascular permeability. The pharmaceutical agent is selected from the group consisting of endostatin, thrombospondin, angiostatin, tumstatin, arrestin, recombinant EPO and polymer conjugated TNP-470. The disease associated with vascular permeability includes, but not limited to, non-proliferative diabetic retinopathy, diabetic nephropathy, nephrotic syndrome, pulmonary hypertension, burn edema, tumor edema, brain tumor edema, IL-2 therapy-associated edema, and other edema-associated diseases.

The invention will be further characterized by the following examples which are intended to be exemplary of the invention.

### EXAMPLES

### Example 1

Effect of Endostatin on Vascular Permeability and Hyperpermeability:

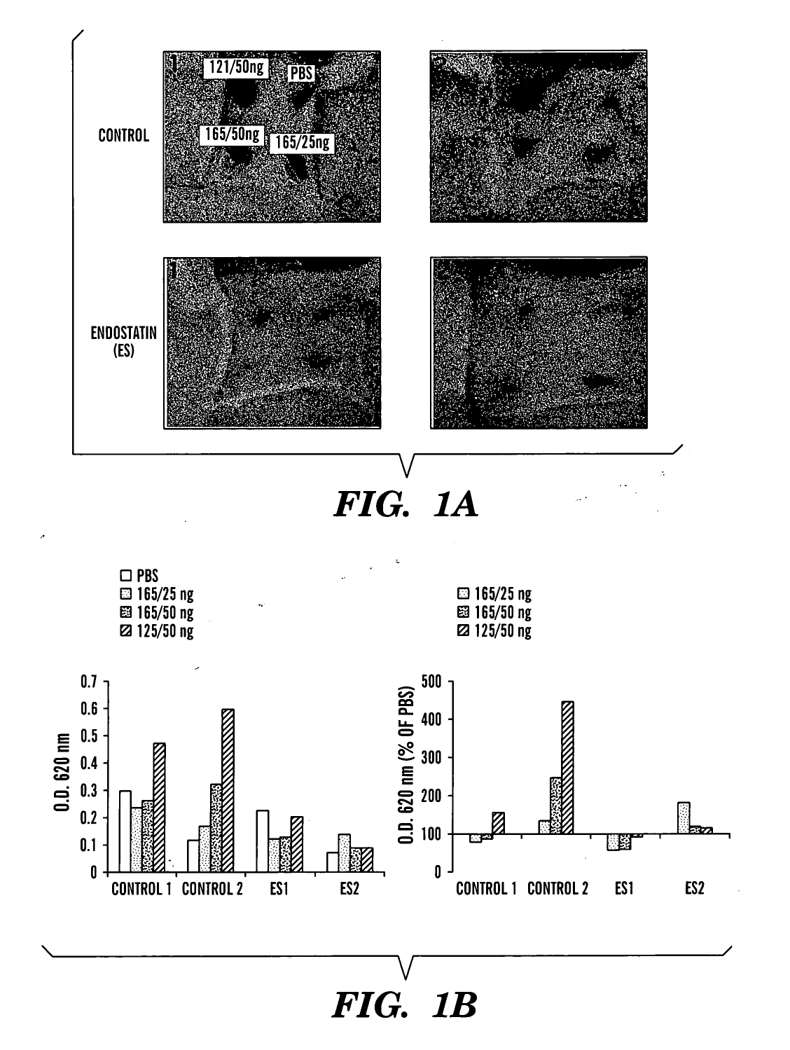

The antiangiogenic factor (endostatin) was injected intraperitoneally to FVB/NJ mice for 4 days. Immediately after the last injection, mice were anasthesized and received intravenous injection of 100 μl Evans Blue dye (1% in PBS). Subsequently, different amounts of VEGF₁₆₅, VEGF₁₂₁ or saline were injected intradermaly. After 20 minutes, mice were sacrificed and skin flap from the back was removed and photographed. Skin samples from the injection sites were excised and incubated in formamide for 5 days in order to extract the dye and O.D. was measured at 620 nm. Macroscopic examination of skin flaps from control mice showed massive extravasation of Evans Blue dye at the VEGF injection sites. VEGF₁₂, had a stronger hyperpermeability activity that VEGF₁₆₅ and there was not much difference between 25 and 50 ng/ml VEGF₁₆₅. Mice treated with the antiangiogenic factor had an overall lower dye leakage than the control and had minor induction of hyperpermeability by VEGF injection. Quantitative analysis of Evans Blue dye extravasation (FIG. 1) confirmed the lower skin capillary permeability of the antiangiogenic factor-treated mice and indicated the weak permeability-inducing effect of VEGF in these mice. These results suggest that the antiangiogenic factor may have a general anti-vascular permeability effects as well as inhibition of VEGF-induced hyperpermeability.

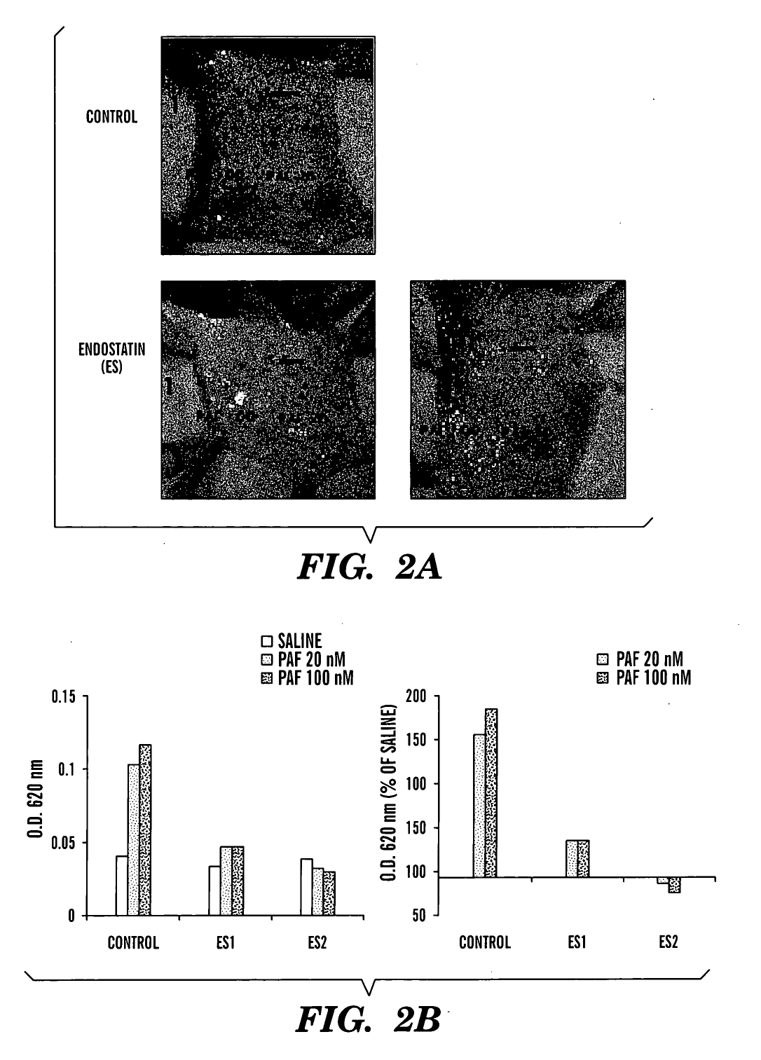

In order to test if the effects of the antiangiogenic factor (endostatin) on vascular permeability is VEGF-specific, we have tested the effects of intradermal injection of platelet-activating factor (PAF) in Nude mice that were previously injected with the antiangiogenic factor and in control mice, as described above. Macroscopic examination of skin flaps confirmed that the antiangiogenic factor inhibits vascular permeability. The antiangiogenic factor also repressed PAF-induced vascular permeability. Quantitative analysis of Evans Blue dye extravasation (FIG. 2) confirmed the lower skin capillary permeability of the antiangiogenic factor-treated mice compared with control and the lack of PAF-induced hyperpermeability in these mice. Thus, it seems that the anti-vascular hyperpermeability effect of the antiangiogenic factor is not restricted to VEGF-induced permeability and affects other mediators of blood vessel permeability such as PAF.

Duration of Exposure to Antiangiogenic Factors to Inhibit Blood Vessel Permeability:

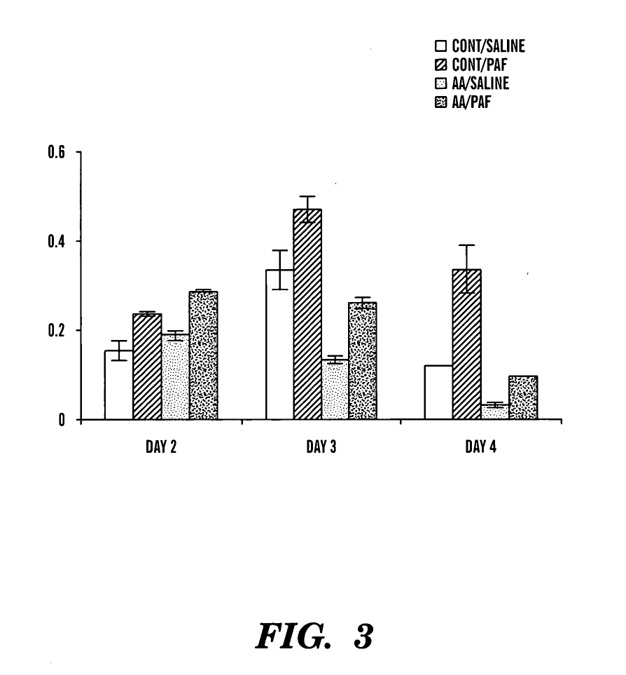

In order to test if continuous exposure to the antiangiogenic factor (endostatin) is required to repress blood vessel permeability, mice (SCID) were anesthetized and “Alzet” pumps loaded with the antiangiogenic factor or saline were implanted intraperitoneally. The pumps release 1 μl the antiangiogenic factor per hour. Skin vessel permeability using Evans Blue dye was performed as described above. Saline and the antiangiogenic factor treated mice were examined 2, 3 and 4 days after pump implantation, as described above (FIG. 3). At day two there was no significant difference between blood vessel permeability in response to PAF injection between saline and the antiangiogenic factor treated mice. In both groups, PAF injection induced higher vessel permeability than saline injection. In contrast, at days three and four both saline and PAF injections in saline treated mice induced significantly higher vessel permeability than in the antiangiogenic factor treated mice. However, in both groups PAF injection induced higher vessel permeability than saline injection. These results indicate that at least 3 days treatment with the antiangiogenic factor were required to reduce skin vessel permeability. Taken together, the results suggest that continuous exposure of the vasculature to the antiangiogenic factor may prevent blood vessel hyperpermeability and leakage of plasma proteins to surrounding tissue. Since the tumor vessels are continuously permeabilized and plasma proteins contained within the tumor support its vascularization the anti-permeability effect of the antiangiogenic factor offers a possible mechanism for its anti-tumor activity.

Endostatin Inhibits Diffusion Through Endothelial Cell Monolayer in Vitro:

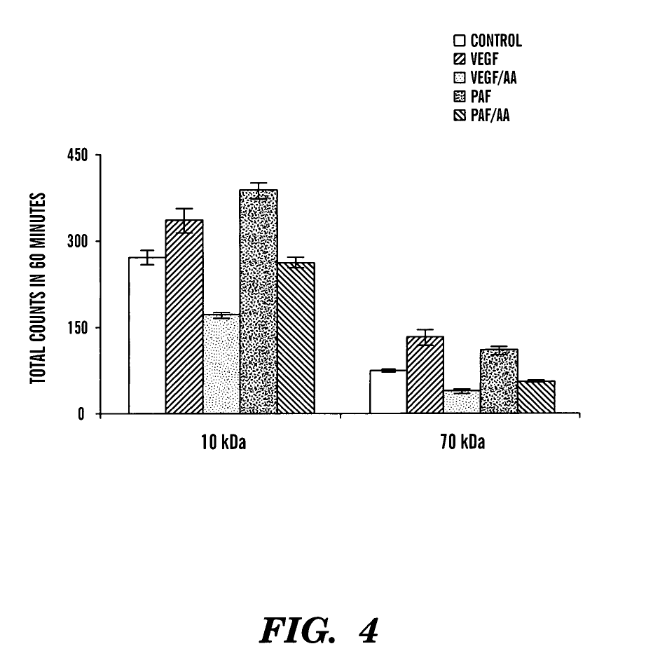

The effects of the antiangiogenic factor (endostatin) on skin vessel permeability in vivo were tested in an in vitro diffusion model designed to mimic blood vessel permeability. Bovine capillary endothelial cells (BCE) were seeded in collagen coated inserts of “Transwells” and grown to confluence. The antiangiogenic factor was added every 24 hours. Four days later the inserts were washed with BCE culture medium and the following tracers and permeability regulators were added to the inserts. Half of the inserts received 5 mg/ml FITC-labeled dextran 10 kDa and the other half received 5 mg/ml FITC-labeled dextran 70 kDa. In addition, some inserts received 50 ng/ml VEGF₁₆₅ or 100 nM PAF. Control inserts received BCE culture medium with fluorescent tracers only. The fluorescence in the lower wells was measured after 10, 20, 30, 45 and 60 minutes by transferring the inserts into new wells. The sum of fluorescent count over 60 minutes showed higher values in cells treated with VEGF₁₆₅ and PAF compared with control cells (FIG. 4). The number of counts in VEGF₁₆₅ and PAF treated cells was observed with 10 kDa and 70 kDa dextrans. Cells that were pre-treated with the antiangiogenic factor showed significantly lower fluorescent counts then control, VEGF₁₆₅-treated and PAF-treated cells in both dextran sizes. The reduction in fluorescent counts in the antiangiogenic factor pre-treated cells was more pronounced in the diffusion of 70 kDa dextran compared with that of 10 kDa dextran. These results indicate that the in vitro diffusion system responds positively to permeability inducing factors such as VEGF and PAF.

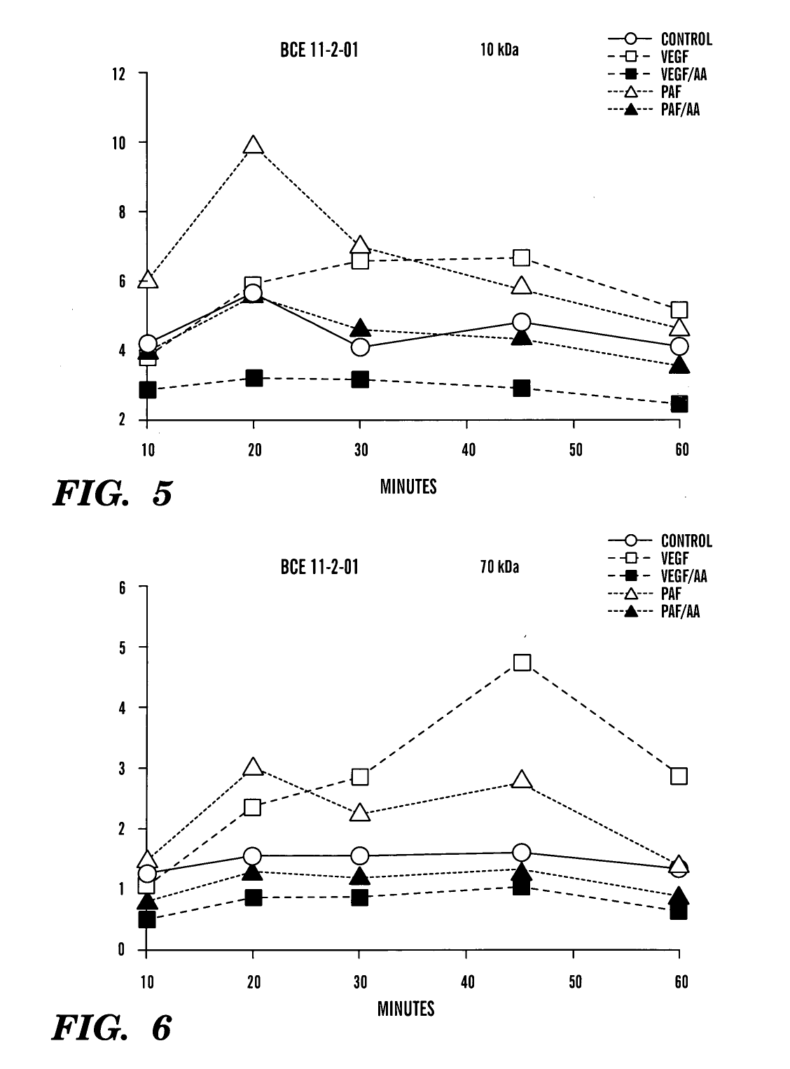

Moreover, the results indicate that the antiangiogenic factor treatment significantly reduces the diffusion of large molecules through EC monolayer. In order to follow the kinetic of the diffusion process, the flow of the tracer was calculated as fluorescent counts per minute (FIGS. 5 and 6). Using 10 kDa dextran (FIG. 5), PAF progressively increased the flow up to 20 minutes and then the flow was reduced and reached similar levels as in the control cells. VEGF₁₆₅ had a similar effect but it reached the maximum flow at 45 minutes and the flow was lower than in PAF-treated cells. In contrast, the flow in control cells was constant and was lower than that observed in PAF and VEGF₁₆₅-treated cells. The results obtained with 70 kDa dextran (FIG. 6) were similar to those of the 10 kDa dextran, only that when using 70 kDa dextran VEGF₁₆₅-treatment resulted in higher flow than in PAF treatment. The antiangiogenic factor pre-treatment resulted in significant reduced flow of the 10 kDa and the 70 kDa dextrans.

Like control cells, the antiangiogenic factor-treated cells had a constant flow during the 60 minutes period. The flow in the antiangiogenic factor-treated cells was lower than that of control cells. Taken together, these results indicate that the antiangiogenic factor treatment results in slower diffusion through EC monolayer. These results suggest that the effect of the antiangiogenic factor on diffusion of large molecules may explain it inhibition of blood vessel permeability. In addition, the in vitro diffusion system can be used to test the effect of anti-angiogenesis and other molecules on blood vessel permeability.

Endostatin Inhibits Swelling of the Lung Tissue

Dilation of the lung tissue may result in lung dysfunction and development of pulmonary hypertension. Mice injected with micro-encapsulated cells producing VEGF (approximately 0.5×10⁶ cells per mouse) developed thickened lung parenchyma 5 days after injection. At a higher magnification we observed generation of several cell layers between the alveoli compared with one layer of cells in mice injected with micro-encapsulated control cells or with micro-encapsulated cells producing endostatin (Endost). In addition, we observed accumulation of extracellular matrix (usually stained pink with H & E staining) in the lung tissue of VEGF-treated mice, suggesting that high levels of circulating VEGF might induce leakage of plasma proteins into the lung tissue. In contrast, the lungs of mice received VEGF producing cells together with endostatin producing cells (0.5×10⁶ encapsulated cells of each) appeared similar to the lungs of mice injected with control cells and had fewer cell layers and no accumulation of extracellular matrix. These results indicate that endostatin may prevent leakage of plasma proteins into the lung tissue and the accumulation of extracellular matrix in the tissue. Moreover, treatment with endostatin reduced the number of cell layers between the alveoli and the lungs of mice that were treated with endostatin appeared similar to control mice. Therefore, endostatin appears to block the swelling of lung tissue and may be used for treatment of pulmonary hypertension.

Endostatin Increases the Assembly of Tight Junction Proteins:

Bovine capillary endothelial cells (BCE) were cultured in the presence of 0.2, 0.5 and 2 μg/ml human endostatin for three days. The cells were fixed and immunostained with anti-β-catenin, occludin, and ZO-1 antibodies (Zymed Laboratories, CA). The staining was developed using FITC-conjugated secondary antibodies and visualized under fluorescent microscopy. Immunostaining for β-catenin marked the cell borders and was more intense when two cells contacted each other. The cell boundary β-catenin staining was intensified in the presence of 0.2 μg/ml endostatin and further intensified in the presence of 0.5 μg/ml endostatin. There was no difference in β-catenin staining between 0.5 and 2.0 μg/ml endostatin. Immunostaining for occludin, in the absence of endostatin, did not show any cell borders demarcation, rather the cell nuclei were stained. However, in the presence of 0.5 and 2.0 μg/ml endostatin cell boundaries were observed mostly when two cells contacted each other. Similar results were obtained with ZO-1 immunostaining. Cells boundaries were only visible in the presence of 0.2-2.0 μg/ml endostatin. These results indicate that immunostaining for tight junction proteins in enhanced in the presence of endostatin and suggest that endostatin may support assembly and stabilization of tight junctions. This is the first documentation of the effects of endostatin on tight junctions that may explain, in part, the mechanism of its antiangiogenic activities. Similar experiments were performed in which BCE were incubated in the presence and absence of 0.5 μg/ml endostatin for 3 days followed by stimulation with PAF for 20 minutes. The cells were fixed and immunostained with anti-α-catenin, occludin, and ZO-1 antibodies (Zymed Laboratories, CA), as described above. PAF treatment significantly reduced the staining intensity of anti-β-catenin, occludin, and ZO-1 only in control cells but not in endostatin-treated cells. These results point to tight junction proteins as possible target for anti-permeability and anti-cancer therapeutic approaches.

The Use of Histamine-Induced Wheal and Flare Assays to Test the Activity of Antiangiogenic Treatment:

Antiangiogenic treatment has entered into clinical trials recently. Molecules that are tested in phase 1 and 2 clinical trials include endostatin, angiostatin, TNP-470, thalidomide, anti-VEGF antibodies, PTK787, SU-5416, SU-6668 and others. Our results indicating that endostatin treatment reduces skin blood vessel permeability support that this test can be used to determine the efficiency of endostatin (and other antiangiogenic agent) treatment in human patients. Mice that received endostatin for several days had lower diffusion of Evans blue from the skin capillaries in response to intradermal VEGF and PAF injection compared with normal mice. The existing test of histamine-induced wheal and flare in skin can be used in order to test bioactivity of endostatin and other antiangiogenic factors. Intradermal injection of histamine leads to the formation of local adema (flare) due to blood vessel hyperpermiability. Humans receiving endostatin and other antiangiogenic factors will have a reduced zone of edema due to the anti-permeability activity. This test will serve as an early surrogate marker for the bioactivity of endostatin and other antiangiogenic factors and help to determine the treatment's efficiency in individual patients.

### Example 2

Synthesis of HPMA Copolymer-TNP-470 Conjugate:

TNP-470 was conjugated to HPMA copolymer-Gly-Phe-Leu-Gly-ethylendiamine via nucleophilic attack on the α-carbonyl on the TNP-470 releasing the chlorine. Briefly, HPMA copolymer-Gly-Phe-Leu-Gly-ethylendiamine (100 mg) was dissolved in DMF (1.0 ml). Then, TNP-470 (100 mg) was dissolved in 1.0 ml DMF and added to the solution. The mixture was stirred in the dark at 4° C. for 12 h. DMF was evaporated and the product, HPMA copolymer-TNP-470 conjugate was redissolved in water, dialyzed (10 kDa MWCO) against water to exclude free TNP-470 and other low molecular weight contaminants, lyophilized and stored at −20° C. Reverse phase HPLC analysis using a C18 column, was used to characterize the conjugate.

BCE Proliferation Assay:

Bovine adrenal capillary endothelial cells were seeded on gelatinized plates (15,000/well). Following 24 h incubation, cells were challenged with free and conjugated TNP-470, and bFGF (1 ng/ml) was added to the medium. Cells were counted after 72 h.

Chick Aortic Ring Assay:

Aortic arches were dissected from day-14 chick embryos and cut into cross-sectional fragments, everted to expose the endothelium, and explanted in Matrigel. When cultured in serum-free MCDB-131 medium, endothelial cells outgrow and three-dimensional vascular channel formation occurred within 2-48 hours. Free and conjugated TNP-470 were added to the culture.

Miles Assay:

One of the problems with angiogenesis-dependent diseases is increased vessel permeability (due to high levels of VPF) which results in edema and loss of proteins. A decrease in vessel permeability is beneficial in those diseases. We have found, using the Miles assay (Claffey, et al., Cancer Res, 56:172-181 (1996)), that free and bound TNP-470 block permeability. Briefly, a dye, Evans Blue (1% in PBS), was injected i.v. to anesthesized mice. After 10 min, human recombinant VEGF₁₆₅ (50 ng/50 μl) was injected intradermally into the back skin. Leakage of protein-bound dye was detected as blue spots on the underside of the back skin surrounding the injection site. After 20 min mice were euthanized. Then, the skin was excised, left in formamide for 5 days to be extracted and the solution read at 620 nm. Putative angiogenesis inhibitors such as free and conjugated TNP-470 were injected daily 3 days (30 mg/kg/day) prior to the VEGF challenge. The same was repeated on tumor-bearing mice to evaluate the effect of angiogenesis inhibitors on tumor vessel permeability.

Hepatectomy:

C57 black male mice underwent a ⅔ hepatectomy through a midline incision after general anesthesia with isoflourane. Free and conjugated TNP-470 (30 mg/kg) was given s.c. every other day for 8 days beginning on the day of surgery. The liver was harvested on the 8^{th} day, weighed and analyzed for histology.

Results:

HPMA copolymer-TNP-470 conjugate was synthesized, purified and characterized by HPLC. Free TNP-470 had a peak at a retention time of 13.0 min while the conjugate had a wider peak at 10.0 min. No free drug was detected following purification.

TNP-470 is not water-soluble but became soluble following conjugation with HPMA copolymer. To evaluate the biological activity of BPMA-TNP-470, the following assays were performed:

BCE proliferation: BCE cell growth was inhibited by TNP-470 and HPMA copolymer-TNP-470 similarly when challenged with bFGF (data not shown).

Aortic ring assay: Free and conjugated TNP-470 reduced the number and length of vascular sprouts and showed efficacy at 50 pg/ml and completely prevented outgrowth at 100 pg/ml. Untreated aortic ring shows abundant sprouting.

Hepatectomy: Following ⅔ hepatectomy, control mice regenerated their resected liver mass to their pre-operative levels (˜1.2 g) by post-operative day 8. Mice treated with free TNP-470 or different doses of its polymer-conjugated form inhibited the regeneration of the liver and retained it at an average size of 0.7 g on post-operative day 8. HPMA-TNP-470 conjugate had a similar effect even when given at a single does on the day of hepatectomy showing a longer circulation time and sustained release from the polymer at the site of proliferating endothelial cells. Because liver regeneration is regulated by endothelial cells growth, it is expected that the same effect will be on proliferating endothelial cells in tumor issue.

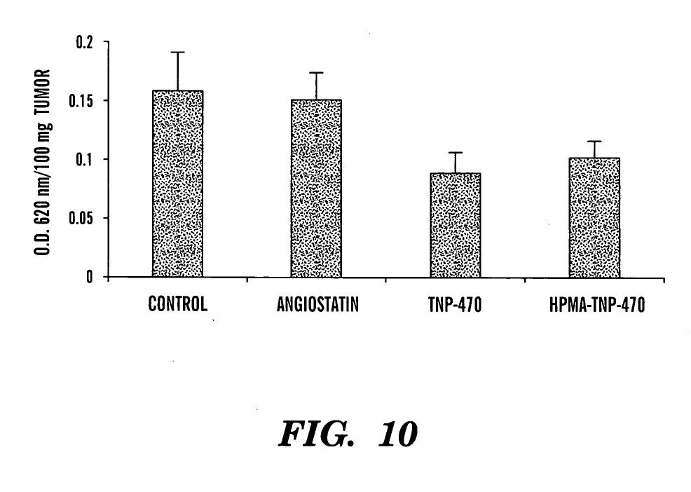

Miles assay: We have compared free and conjugated TNP-470 to other angiogenesis inhibitors in the Miles assay. We have found that free TNP-470 and HPMA copolymer-TNP-470 had similar inhibitory effect on VEGF induced vessel permeability as opposed to the control groups and indirect angiogenesis inhibitors such as Herceptin and Thalidomide. Free and conjugated TNP-470 at 30 mg/kg/day for three days also decreased tumor vessel permeability in A2058 human melanoma-bearing mice (FIG. 10).

Conclusions:

HPMA copolymer-TNP-470 inhibited the proliferation of BCE cells and chick aortic rings in vitro. In vivo the conjugate had a similar effect as the free TNP-470 on liver regeneration following hepatectomy. This suggests that it retained its inhibitory activity when released from the polymeric conjugate by lysosomal enzymatic cleavage of the tetrapeptide (Gly-Phe-Leu-Gly) linker in the proliferating endothelial cells.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents (FIG. 10).

### REFERENCES

The references cited below and incorporated throughout the application are incorporated herein by reference.
1. Ingber D et al, Nature 348, 555-557 (1990).
2. Kudelka et al., New Engl J Med 338, 991-992 (1998).
3. Folkman J., Angiogenesis, In Harrison's Textbook of Internal Medicine 15^{th} Edition, edited by E. Braunwald et al., McGraw Hill, pp. 517-530 (2001).
4. R. Duncan, J. K. Coatsworth, S. Burtles, Hum Exp Toxicol 17, 93-104 (1998).
5. H. Maeda, J. Wu, T. Sawa, Y. Matsumura, K. Hori, J Controlled Release 65, 271-84 (2000).
6. Claffey et al., Cancer Res 56, 172-181 (1996).
7. Andriopoulou, P., Navarro, P., Zanetti, A., Lampugnani, M. G., and Dejana, E. (1999). Histamine induces tyrosine phosphorylation of endothelial cell-to-cell adherens junctions., Arterioscler Thromb Vasc Biol 19, 2286-97.
8. Antonetti, D. A., Barber, A. J., Hollinger, L. A., Wolpert, E. B., and Gardner, T. W. (1999). Vascular endothelial growth factor induces rapid phosphorylation of tight junction proteins occludin and zonula occluden 1. A potential mechanism for vascular permeability in diabetic retinopathy and tumors., J Biol Chem 274, 23463-7.
9. Antonetti, D. A., Barber, A. J., Khin, S., Lieth, E., Tarbell, J. M., and Gardner, T. W. (1998). Vascular permeability in experimental diabetes is associated with reduced endothelial occludin content: vascular endothelial growth factor decreases occludin in retinal endothelial cells. Penn State Retina Research Group., Diabetes 47, 1953-9.
10. Archer, C. B. (1993). Platelet activating factor—a mediator of inflammation in the skin—medical implications., Clin Exp Dermatol 18, 489-95.
11. Bussolino, F., and Camussi, G. (1995). Platelet-activating factor produced by endothelial cells. A molecule with autocrine and paracrine properties., Eur J Biochem 229, 327-37.
12. Clough, G. F., Boutsiouki, P., and Church, M. K. (2001). Comparison of the effects of levocetirizine and loratadine on histamine-induced wheal, flare, and itch in human skin., Allergy 56, 985-8.
13. Connolly, D. T., Heuvelman, D. M., Nelson, R., Olander, J. V., Eppley, B. L., Delfino, J. J., Siegel, N. R., Leimgruber, R. M., and Feder, J. (1989a). Tumor vascular permeability factor stimulates endothelial cell growth and angiogenesis., J Clin Invest 84, 1470-8.
14. Connolly, D. T., Olander, J. V., Heuvelman, D., Nelson, R., Monsell, R., Siegel, N., Haymore, B. L., Leimgruber, R., and Feder, J. (1989b). Human vascular permeability factor. Isolation from U937 cells., J Biol Chem 264, 20017-24.
15. Dejana, E. (1997). Endothelial adherens junctions: implications in the control of vascular permeability and angiogenesis., J Clin Invest 100, S7-10.
16. Dvorak, H. F. (1986). Tumors: wounds that do not heal. Similarities between tumor stroma generation and wound healing., N Engl J Med 315, 1650-9.
17. Dvorak, H. F., Brown, L. F., Detmar, M., and Dvorak, A. M. (1995). Vascular permeability factor/vascular endothelial growth factor, microvascular hyperpermeability, and angiogenesis., Am J Pathol 146, 1029-39.
18. Esser, S., Wolburg, K., Wolburg, H., Breier, G., Kurzchalia, T., and Risau, W. (1998). Vascular endothelial growth factor induces endothelial fenestrations in vitro., J Cell Biol 140, 947-59.
19. Fischer, S., Clauss, M., Wiesnet, M., Renz, D., Schaper, W., and Karliczek, G. F. (1999). Hypoxia induces permeability in brain microvessel endothelial cells via VEGF and NO., Am J Physiol 276, C812-20.
20. Fischer, S., Wobben, M., Marti, H. H., Renz, D., and Schaper, W. (2002). Hypoxia-Induced Hyperpermeability in Brain Microvessel Endothelial Cells Involves VEGF-Mediated Changes in the Expression of Zonula Occludens-1., Microvasc Res 63, 70-80.
21. Martin, T. A. (2001). Tight junctions and their role in cancer metastasis., Histol Histopathol 16, 1183-95.
22. Murohara, T., Horowitz, J. R., Silver, M., Tsurumi, Y., Chen, D., Sullivan, A., and Isner, J. M. (1998). Vascular endothelial growth factor/vascular permeability factor enhances vascular permeability via nitric oxide and prostacyclin., Circulation 97, 99-107.
23. Roberts, W. G., and Palade, G. E. (1995). Increased microvascular permeability and endothelial fenestration induced by vascular endothelial growth factor., J Cell Sci 108, 2369-79.
24. Saria, A., and Lundberg, J. M. (1983). Evans blue fluorescence: quantitative and morphological evaluation of vascular permeability in animal tissues., J Neurosci Methods 8, 41-9.
25. Thurston, G., Suri, C., K, S., J, M., T N, S., G D, Y., and D M, M. (1999). Leakage-resistant blood vessels in mice transgenically overexpressing angiopoietin-1., Science 286, 2511-4.
26. Wang, W., Dentler, W. L., and Borchardt, R. T. (2001). VEGF increases BMEC monolayer permeability by affecting occludin expression and tight junction assembly., Am J Physiol Heart Circ Physiol 280, H434-40.

1. A method for assessing bioeffectiveness of an antiangiogenic compound in a patient being treated with said compound comprising:
a) administering to said patient an intradermal injection of histamine before treating the patient with the antiangiogenic compound and measuring a histamine-induced local edema;
b) treating the patient with the antiangiogenic compound; and
c) administering to said patient an intradermal injection of histamine subsequent to treating the patient with the antiangiogenic compound and measuring the histomine-induced local edema, wherein a decrease in measurement of the histamine-induced local edema compared to that seen before the treatment with the antiangiogenic compound indicates that the compound is bioeffective.
